(19) United States
(12) Patent Application Publication
(10) Pub. No.: US 2010/0158882 A1
(43) Pub. Date: Jun. 24, 2010

(54) IMMUNOMODULATING PROBIOTIC LACTIC ACID BACTERIA

(75) Inventors: Peter Frederik Zuurendonk, Leiden (NL); Michiel Kleerebezem, Ede (NL); Johannes Snel, Wageningen (NL); Maria Marco, Renkum (NL); Hubertus Franciscus Jozef Savelkoul, Bennekom (NL)

Correspondence Address: FOLEY AND LARDNER LLP;SUITE 500, 3000 K STREET NW, WASHINGTON, DC, 20007, US

(73) Assignee: Campina Nederland Holding B.V.

(21) Appl. No.: 12/520,465

(22) PCT Filed: Dec. 21, 2007
(86) PCT No.: PCT/NL2007/050694

§ 371 Date: Dec. 14, 2009

(30) Foreign Application Priority Data
Dec. 22, 2006 (EP) 06127118.5

(51) Int. Cl. A61K 35/74; A23C 9/12; C12N 1/20; A61P 37/08

(52) U.S. Cl. 424/93.45; 426/61; 435/252.9

(57) ABSTRACT

The present invention relates to immuno modulating probiotic lactic acid bacteria, to methods wherein the bacteria are used to reduce allergy, and to food products wherein the bacteria may be in incorporated to reduce allergy upon consumption of the product. Preferred probiotic lactic acid bacteria stimulate the Th1 and/or Th3 responses and/or represses Th2 responses as may be determined by the cytokine profiles that are induced in human peripheral blood mononuclear cells upon coincubation with the lactic acid bacteria.

### FIELD OF THE INVENTION

The present invention relates to immunomodulating probiotic lactic acid bacteria, to methods wherein the bacteria are used to reduce allergy, and to food products wherein the bacteria may be incorporated to reduce allergy upon consumption of the product.

### BACKGROUND OF THE INVENTION

IgE-mediated allergy, also referred to as type I hypersensitivity, is the most important hypersensitivity reaction in the body. It is induced by a strong reaction against certain types of environmental compounds (food molecules, pollen, house dust, bee venom, etc) referred to as allergens. Typical type I hypersensitivities associated with IgE antibodies include hay fever, asthma, urticaria, anaphylaxy shock and the like.

Exposure to allergens can result in induction of either immunological tolerance or an active immune response. T-lymphocytes play an important role in directing the immune response. Different subsets of T-lymphocytes can be found in the human body. Allergy is the result of a T-helper type 2-(Th2)-mediated immune response and is characterized by the production of interleukins IL-4, IL-5 and IL-13. IL-4 is the cytokine responsible for the formation of IgE-producing cells. Allergy has long been seen as a unbalance between Th2 and Th1-(T-helper type 1) responses, Therefore, it was thought that stimulation of a Th1 response should result in a lowering of a Th2 response. However, recently published evidence suggests that Th2 responses are physiologically normal responses that can exist in the absence of allergy, as well as in the presence of strong Th1 responses. Therefore, a role for T regulatory cells (Treg or Th3) and induction of tolerance has been postulated to control the development of allergy.

Allergens do not directly target T-lymphocytes, but are phagocytized by antigen presenting cells such as dendritic cells that can be found throughout the intestinal tract, the respiratory tract and underneath the skin Antigen presenting cells process the allergens and express parts of it at their cell surface. These are recognized by T lymphocytes in association with other cell surface markers such as MHC-II and B7-2 molecules.

The response of the T lymphocytes is dependent on the activation state of the antigen presenting cells as well as the cytokines produced by these cells. Non-activated dendritic cells are recognized by a low expression of the surface markers MHC-II and B7-2. These cells produce IL-10, and stimulate regulatory T cells (also called T-suppressor cells or T-helper 3 cells; Th3 cells) leading to tolerance induction. Activated dendritic cells have increased expression of MHC-II and B7-2. These cells may produce high levels of IL-12 and IFN-γ that stimulate formation of T-helper 1 cells (Th1 cells), or produce IL-6 and IL-10 to stimulate T-helper 2 cells (Th2 cells). The latter cell type may lead to allergy induction.

The gastro-intestinal tract contains the largest outer surface area of the human body, and has a dense concentration of non-self molecules, such as food molecules and commensal bacteria, that is in close contact with the locally present mucosal immune system. It is thus not surprising that the intestinal microbiota play a role in the development of allergy. Indeed different colonization patterns have been found in allergic children compared to normal healthy children. Lactobacilli are a major component of the commensal microflora of humans and are frequently used as probiotics. Therefore Lactobacilli have been investigated for their ability to play a role in the prevention of allergy.

Kalliomaki et al. (Lancet, 2001. 357(9262): p. 1076-9) reported that Lactobacillus GG when given either to bottle fed newborns or to their mothers around the time of birth and during the next few months significantly reduced the chance of these infants of developing a cow's milk allergy. Even at the age of four years, the protective effect could still be observed (Kalliomaki et al., Lancet, 2003. 361(9372): p. 1869-71). However, these studies have focussed on allergy prevention in newborns and do not relate to a reduction of symptoms in patients with an existing allergy. Indeed, in contrast to the promising effects on allergy prevention, no effect of Lactobacillus GG on birch-pollen allergy was observed (Helin et al., Allergy, 2002. 57(3): p. 243-6).

EP1364586 discloses lactic acid bacterial strains belonging to the Lactobacillus genus, and in particular Lactobacillus paracasei strains that are able to promote the induction of oral tolerance in mice to the cow's milk protein beta-lactoglobulin.

US2005214270 discloses anti-allergic agents comprising as an active ingredient one or more lactic acid bacterial strains of the species Lactobacillus acidophilus or Lactobacillus fermentum that suppress the IgE level in an ovalbumin hypersensitized mouse model.

Ishida et al. examined the efficacy of orally administered Lactobacillus acidophilus strain L-92 on perennial allergic rhinitis (J Dairy Sci, 2005. 88(2): p. 527-33.) and on symptoms of Japanese cedar pollen allergy (Biosci Biotechnol Biochem, 2005. 69(9): p. 1652-60). Although significant improvement of clinical symptoms such as nasal and ocular symptom-medication scores was observed, no significant differences in serum antihouse dust mite IgE levels or in Th 1/Th 2 ratio between the 2 groups were seen.

EP1634600 discloses anti-allergic compositions comprising as active ingredient the Lactobacillus paracasei KW 3110 strain that induces IL 12 production and reduces IL 4 production in vitro in mouse spleen lymphocytes sensitized with ovalbumin.

Several reports have been published of human peripheral blood mononuclear cells (PBMCs) being used to screen Lactobacillus cultures for their immunomodulating capabilities (Niers et al., Clin Exp Allergy, 2005. 35(11): p. 1481-9; Miettinen et al., Infect Immun, 1998. 66(12): p. 6058-62; Miettinen et al., Infect Immun, 1996. 64(12): p. 5403-5).

There is however still a need for further probiotic lactic acid bacterial strains with immunomodulating capabilities that may be used in methods for reducing allergy, and that may e.g. be in incorporated into food products to reduce allergy.

### DESCRIPTION OF THE INVENTION

In a first aspect the present invention relates to lactic acid bacterial strains with immunomodulating capabilities. The lactic acid bacterial strains may be used as a medicament, preferably they may be used in the treatment of allergy. The term “lactic acid bacteria” is used herein to refer to bacteria, which produce lactic acid as a product of fermentation, including e.g. bacteria of the genus Lactobacillus, Streptococcus, Lactococcus, Oenococcus, Leuconostoc, Pediococcus, Carnobacterium, Propionibacterium, Enterococcus and Bifidobacterium. The lactic acid bacterial strains of the invention are “probiotics” or “probiotic strains”, which term herein refers to a strain of live bacteria, which have a beneficial effect on the host when ingested (e.g. enterally or by inhalation) by a subject. A “subject” refers herein to a human or non-human animal, in particular a vertebrate.

The allergy that may be treated by the lactic acid bacterial strains of the invention preferably is an IgE-mediated allergy, also referred to as type I hypersensitivity. The treatment may comprise the prevention and/or reduction of environmental allergies such as hay fever, topic dermatitis, bronchial asthma, chronic allergic rhinitis, urticaria, and anaphylactic shock. The allergy to be treated may be caused by a large variety of allergens including: Airborne particles (hay fever): (pollen from) grass, weeds, timothy grass, birch trees and mold spores; Drugs: penicillin, sulfonamides, salicylates (also found naturally in numerous fruits) and local anaesthetics; Foods (food allergy): nuts, peanuts, sesame, seafood, eggs (typically albumen,), peas, beans, soybeans and other legumes, celery and celeriac, soy, milk, wheat (gluten) and corn or maize; Insect stings: bee sting venom and wasp sting venom; and Animal products (animal allergy): animal hair and dander, cockroach calyx, and dust mite excretion.

The lactic acid bacterial strains that are used as active ingredient in the treatment of allergy have immunomodulating capabilities. Preferably, the lactic acid bacterial strains have one or more of the following immunomodulating capabilities that are advantageous in the treatment of allergy:
a) stimulation of T-helper 1 cells (Th1 cells) and/or a Th1 response;
b) stimulation of T-helper 3 cells (Th3 cells) and/or a Th3 response. Th3 cells are also referred to as regulatory T cells or T-suppressor cells and favor induction of tolerance;
c) absence of, or reduced induction of an inflammatory response and,
d) absence of stimulation and/or repression of T-helper 2 cells (Th2 cells) and/or a Th2 response, which may lead to induction of allergy.

In accordance with the invention, a lactic acid bacterium with the capability to stimulate T-helper 1 cells (Th1 cells) and/or a Th1 response preferably is a lactic acid bacterium that upon co-incubation in vitro with human PBMCs induces IL-12 and/or IFN-γ production. Preferably, the lactic acid bacterium induces at least 50, 60, 70, 80 or 90% of the IL-12 production that is induced in human PBMCs in vitro by at least one of four reference strains selected from L. plantarum BI-1, L. plantarum BI-2, L. fermentum BI-6 and L. plantarum BI-3, of which L. plantarum BI-1 and L. plantarum BI-2 are more preferred. Preferably, the lactic acid bacterium further induces at least 40, 50, 60, 70, 80 or 90% of the IFN-γ production that is induced in human PBMCs in vitro by at least one of the four reference strains from L. plantarum BI-1, L. plantarum BI-2, L. fermentum BI-6 and L. plantarum BI-3, of which L. plantarum BI-1 and L. plantarum BI-2 are more preferred. Preferably, the lactic acid bacterium upon incubation in vitro with human PBMCs further induces limited amounts of pro-inflammatory and/or pro-Th2 response cytokines such as e.g. IL-1β. Thus, a preferred lactic acid bacterium of the invention upon incubation in vitro with human PBMCs further induces no more than 150, 140, 130, 120 or 110% of the amount of IL-1β as is induced under the same conditions by at least one of the four reference strains (BI-1, BI-2, BI-3 and BI-6). A particularly preferred lactic acid bacterium with the capability to stimulate T-helper 1 cells and/or a Th1 response is a bacterium selected from L. plantarum BI-1, L. plantarum BI-2, L. fermentum BI-6 and L. plantarum BI-3. L. plantarum BI-1, L. plantarum BI-2, L. fermentum BI-6 and L. plantarum BI-3 were deposited on 18 Dec. 2006 by NIZO Food Research, Ede, the Netherlands under the Budapest Treaty at the Centraal Bureau voor Schimmelcultures, Baarn, The Netherlands. Strains were assigned the following deposit no.'s L. plantarum BI-1: CBS120663; L. plantarum BI-2: CBS120664; L. fermentum BI-6: CBS120661; and L. plantarum BI-3: CBS120662. BI-3 appeared to be initially incorrectly typed as a L. fermentum, but it was later re-typed.

A further preferred lactic acid bacterium with the capability to stimulate T-helper 1 cells (Th1 cells) and/or a Th1 response preferably is a lactic acid bacterium that upon co-incubation in vitro with human PBMCs from allergic individuals, more preferably from individuals with a birch pollen allergy, induces IL-12. Preferably, the lactic acid bacterium induces at least 50, 60, 70, 80 or 90% of the IL-12 production that is induced in human PBMCs in vitro by at least one of three reference strains selected from L. plantarum BI-1, L. plantarum BI-2 and L. fermentum BI-6.

In accordance with the invention, a lactic acid bacterium with the capability to stimulate T-helper 3 cells (Th3 cells) and/or a Th3 response preferably is a lactic acid bacterium that upon co-incubation in vitro with human PBMCs induces IL-10 production. Preferably, the lactic acid bacterium induces at least 50, 60, 70, 80 or 90% of the IL-10 production that is induced in human PBMCs in vitro by at least one of the four reference strains L. plantarum BI-1, L. plantarum BI-2, L. fermentum BI-6 and L. plantarum BI-3, of which L. fermentum BI-6 and L. plantarum BI-3 are more preferred.

A further preferred lactic acid bacterium with the capability to stimulate T-helper 3 cells (Th3 cells) and/or a Th3 response is a lactic acid bacterium that upon co-incubation in vitro with human PBMCs, preferably PBMCs from an allergic individual, more preferably from an individual with an IgE-mediated allergy, most preferably from an individual with a pollen allergy (of which birch pollen are most preferred), induces IL-10 production. Preferably, the lactic acid bacterium induces at least 50, 60, 70, 80 or 90% of the IL-10 production that is induced in vitro in human PBMCs from an allergic individual, more preferably from individuals with a birch pollen allergy, by at least one of the four reference strains L. plantarum BI-1, L. plantarum BI-2, and L. fermentum BI-6 L. plantarum BI-3, of which L. plantarum BI-3 is more preferred.

In accordance with the invention, a lactic acid bacterium with the capability to repress and/or at least lacking the capability to stimulate T-helper 2 cells (Th2 cells) and/or a Th2 response (leading to induction of allergy) preferably is a lactic acid bacterium that upon co-incubation in vitro with human PBMCs represses or at least does not stimulate the induction of IL-13. Preferably, the lactic acid bacterium induces no more than 150, 140, 130, 120 or 110% of the amount of IL-13 as is induced under the same conditions by at least one of the three reference strains (BI-1, BI-2 and BI-6). Preferably the lactic acid bacterium represses or at least does not stimulate the induction of IL-13 upon co-incubation in vitro with human PBMCs, more preferably from an individual with an IgE-mediated allergy, most preferably from an individual with a pollen allergy (of which birch pollen are most preferred).

The levels of cytokines as induced upon co-incubation in vitro of the above described lactic acid bacteria of the invention with human PBMCs may be performed by routine methods known in the art. Preferably cytokine induction by lactic acid bacteria is determined as described in the Examples herein: human PBMCs are Ficoll-Paque purified from human buffy coat and resuspended in 2 ml RPMI 1640 medium containing 10% heat-inactivated foetal calf serum, 2 mM glutamine, and the antibiotics penicillin and streptomycin, at a concentration of 10⁶/ml PBMCs are incubated with the lactic acid bacterium to be tested at a concentration of 10⁶ cfu/ml at 5% CO₂ and 37° C. for 24 hours. Cytokines as indicated above are then determined in culture supernatants using routine methods. More preferably cytokines are determined after 48, 72 or 96 hours. Most preferably cytokines are determined PBMC cultures that have been stimulated with LPS or antibodies against CD3 and/or CD28. Human PBMCs may be obtained from blood donated by health volunteers, volunteers with an allergy caused by any of a large variety of allergens as described above, including e.g. birch pollen.

The term IL-12 is typically used herein to refer to a collection of IL-12 related proteins. The preferred form of IL-12 that is determined in the cytokine assays described herein is the bioactive form of IL-12, IL12p70, which is a 75 kDa heterodimer comprised of independently-regulated disulfide-linked 40 kDa (p40) and 35 kDa (p35) subunits.

The lactic acid bacterial strains of the invention preferably are of the genus Lactobacillus. The bacteria should be food-grade, i.e. they should be considered as not harmful, when ingested by a human or animal subject. It is understood that non-food grade bacteria, for example pathogenic bacteria, which have been modified so that they are no longer harmful when ingested by a subject, are included within the scope of the invention. The Lactobacillus strains may be of the following species: L. rhamnosus, L. casei, L. paracasei, L. helveticus, L. delbrueckii, L. reuteri, L. brevis, L. crispatus, L. sakei, L. jensenii, L. sanfransiscensis, L. fructivorans, L. kefiri, L. curvatus, L. paraplantarum, L. kefirgranum, L. parakefir, L. fermentum, L. plantarum, L. acidophilus, L. johnsonii, L. gasseri, L. xylosus, L. salivarius etc. Preferred species of lactic acid bacterial strains are of a species selected from L. acidophilus, L. plantarum, L. fermentum, L. rhamnosus, L. paracasei, L. acetotolerans, L. reuteri, L. casei, and L. paracasei subsp., more preferred are L. plantarum and L. fermentum, most preferred are the deposited reference strains as defined herein above. A lactic acid bacterial strain of the present invention further preferably is resistant to digestive juice such as gastric acid, bile acid so as allow the bacteria to reach the intestinal tract alive (in case they are not encapsulated/protected). Moreover, a preferred lactic acid bacterial strain is highly adhesive to intestinal tract so that it remains in the intestinal tract for longer periods of time and may colonise the intestinal tract.

It is understood that replicates and/or derivatives of the deposited strains or any other strain according to the invention are encompassed by the invention. The term “replicate” refers to the biological material that represents a substantially unmodified copy of the material, such as material produced by growth of micro-organisms, e.g. growth of bacteria in culture media. The term “derivative” refers to material created from the biological material and which is substantially modified to have new properties, for example caused by heritable changes in the genetic material. These changes can either occur spontaneously or be the result of applied chemical and/or physical agents (e.g. mutagenesis agents) and/or by recombinant DNA techniques as known in the art. When referring to a strain “derived” from another strain, it is understood that both “replicates” of that strain, as well as “derivatives” of the strain are encompassed, as long as the derived strain still retains the immunomodulating capabilities of the strain from which it was derived, and therefore can be used in the treatment of allergy.

In another aspect the invention relates to the use of a lactic acid bacterial strain for the preparation (manufacture) of a composition for the treatment allergy as defined herein above. The composition that is manufactured using one or more strain(s) according to the invention may be any type of composition, which is suitable for consumption by, or administration to a subject, preferably a human subject suffering from an allergy as defined above. The composition may be a food, a food supplement composition, a nutraceutical or a pharmaceutical composition. Depending on the type of composition and its preferred administration method, the components and texture of the composition may vary. A food or food/nutritive composition comprises besides the bacterial strain(s) of the invention also a suitable food base. A food or food composition is herein understood to include solids (for example powders), semi-solids and/or liquids (e.g. a drink or beverage) for human or animal consumption. A food or food/nutritive composition may be a dairy product, such as fermented dairy products, yoghurt, milk or milk-based drinks, buttermilk, yoghurt-based drinks, cheese and butter, ice-cream, desserts (e.g. milk-derived desserts like custards, Dutch equivalent: “vla”), soft curd cheese (Dutch equivalent: “kwark”), fresh cheese (e.g. cottage cheese), etc. Such foods or food compositions may be prepared in a manner known per se, e.g. by adding the strain(s) of the invention to a suitable food or food base, in a suitable amount (see e.g. WO 01/82711). In a further embodiment, the strain(s) are used in or for the preparation of a food or food/nutrient composition, e.g. by fermentation. Examples of such strains include probiotic lactic acid producing bacteria of the invention. In doing so, the strain(s) of the invention may be used in a manner known per se for the preparation of such fermented foods or food/nutrition compositions, e.g. in a manner known per se for the preparation of fermented dairy products using lactic acid producing bacteria. In such methods, the strain(s) of the invention may be used in addition to the micro-organism usually used, and/or may replace one or more or part of the micro-organism usually used. For example, in the preparation of fermented dairy products such as yoghurt or yoghurt-based drinks, a live food grade lactic acid producing bacterium of the invention may be added to or used as part of a starter culture or may be suitably added during or after such a fermentation. Also flavourings, anti-oxidants, vitamins, minerals, colouring agents, etc may be present. Although preferably living cells are used, dead or non-viable cells may also be used in some compositions.

A preferred lactic acid bacterial strain of the invention is a strain that is stable in a dairy product, particularly in a fermented dairy product. Stability of the strain is herein understood as survival of viable bacteria after prolonged storage in a (fermented) dairy product. Preferably a lactic acid bacterial strain of the invention is stable in a (fermented) dairy product under refrigeration conditions (4-6° C.) for at least 1, 2, 3, or 4 weeks whereby it is understood that the survival log of the bacterial strain (i.e. cfu after storage/cfu at start) is at least −2, −1.3, −1.2, −0.5, −0.2, or −0.1. An example of conditions for determining stability or survival rate is described in Example 3 herein.

Apart from an effective amount of one or more of the lactic acid bacterial strains of the invention, a food supplement may comprise one or more carriers, stabilizers, prebiotics and the like. Preferably, the composition is in powder form, for enteral (preferably oral) administration, although nasal administration or inhalation may also be suitable. When using living cells of the strain(s), the cells may be present in an encapsulated form in order to be protected against the stomach juices which means that in this case the cells do not need to be resistant to digestive juice. The composition may e.g. be in the form of a powder packed in a sachet which can be dissolved or dispersed in water, fruit juice, milk or another beverage. The dose of cells per strain is preferably at least 1×10⁶ cfu per strain, preferably between about 1×10⁶-1×10¹² cfu (colony forming units) per day, more preferably between about 1×10⁷-1×10¹¹ cfu/day, more preferably about 1×10⁸-5×10¹⁰ cfu/day, most preferably between 1×10⁹-2×10¹⁰ cfu/day. The effective dose may be provided as a single dosage or may be subdivided into several smaller dosages and administered for example in two, three or more portions per day.

Apart from one or more of the lactic acid bacterial strains of the invention, a nutritional composition preferably comprises carbohydrates and/or proteins and/or lipids suitable for human and/or animal consumption. The compositions may or may not contain other bioactive ingredients, such as other (probiotic) strains, and prebiotics, which support the probiotic strains. When using living cells of the strain(s), the cells may be present in an encapsulated form in order to be protected against the stomach juice. The dose of living cells per strain is preferably at least 1×10⁶ cfu, preferably between about 1×10⁶-1×10¹² cfu (colony forming units) per day, more preferably between about 1×10⁷-1×10¹¹ cfu/day, more preferably about 1×10⁸-5×10¹⁰ cfu/day, most preferably between 1×10⁹-2×10¹⁰ cfu/day. The nutrition composition may replace the normal food/drink intake of a subject, or may be consumed in addition thereto.

It is understood that when dead or non-viable cells dosages equivalent to the above cfu's are used. These equivalent dosages may be e.g. be determined using optical density (e.g. OD₆₀₀) or by protein or DNA quantification.

One or more of the lactic acid bacterial strains of the invention in a suitable dosage may also be used to make a nutraceutical or pharmaceutical composition for treatment, therapy or prophylaxis of allergy. Nutraceutical/pharmaceutical compositions will usually be used for enteral, for example oral application. Nutraceutical/pharmaceutical compositions will usually comprise a pharmaceutical carrier in addition to the strain(s) of the invention. The preferred form depends on the intended mode of administration and (therapeutic) application. The pharmaceutical carrier can be any compatible, non-toxic substance suitable to deliver the strains(s) to the desired body cavity, e.g. the intestine of a subject. E.g. sterile water, or inert solids may be used as the carrier usually complemented with pharmaceutically acceptable adjuvants, buffering agents, dispersing agents, and the like. Nutraceutical/pharmaceutical compositions may further comprise additional biologically or pharmaceutically active ingredients.

It is understood that in the method, uses and compositions of the invention, at least two or more strains may be combined in one composition or co-administered to a subject. The strains may be present in different compositions and only combined in vivo after administration of the different compositions to a subject. Alternatively the strains may be present in a single composition. In both cases the administration of two or more strains is referred to as “co-administration”.

In a preferred embodiment, at least one strain having the capability to stimulate T-helper 3 cells (Th3 cells) and/or a Th3 response as defined herein is combined with at least one strain having the capability to induce no or only a reduced inflammatory response as defined herein, in the methods, uses and composition of the invention. Alternatively, at least one strain having the capability to stimulate T-helper 3 cells (Th3 cells) and/or a Th3 response as defined herein may be combined with at least one strain having the capability to stimulate T-helper 1 cells (Th1 cells) and/or a Th1 response as defined herein, or at least one strain having the capability to induce no or only a reduced inflammatory response as defined herein may be combined with at least one strain having the capability to stimulate T-helper 1 cells (Th1 cells) and/or a Th1 response as defined herein, in the methods, uses and composition of the invention. In yet another preferred embodiment at least one strain having the capability to repress and/or at not stimulate T-helper 2 cells (Th2 cells) and/or a Th2 response as defined herein is combined with at least one strain having the capability to stimulate T-helper 3 cells (Th3 cells) and/or a Th3 response as defined and/or is combined with at least one strain having the capability to induce no or only a reduced inflammatory response as defined herein, and/or with at least one strain having the capability to stimulate T-helper 1 cells (Th1 cells) and/or a Th1 response as defined herein, in the methods, uses and composition of the invention. Such combinations of strains are in some instance superior over administration of only strain(s) having one of the indicated immunomodulating capabilities.

In yet a further aspect, the invention pertains to a method for preparing a composition for the treatment of allergy, comprising the steps of: a) growing at least one lactic acid bacterial strain as defined herein in a suitable liquid or solid medium; b) optionally isolating the strain from the medium, for example by centrifugation and/or filtration and performing down stream processing as known in the art, for example lyophilisation, spray drying and/or freezing; and, c) formulating the strain into a form suitable for administration to a subject. The strains of the invention may be grown on artificial media or on natural media, such as (low fat) milk, yoghurt, and the like. It may then be used directly to make a composition according the invention, or the bacteria may be concentrated or isolated by centrifugation and/or filtration from the medium and then formulated into suitable compositions.

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the element is present, unless the context clearly requires that there be one and only one of the elements. The indefinite article “a” or “an” thus usually means “at least one”.

### EXAMPLES

### Example 1

### Effects of Lactobacilli on Cytokine Production by Human PBMC

### 1.1 Materials and Methods

1.1.1 One-Day (a) and Four-Day (b) In Vitro Characterisation were Carried Out Using the Procedure Referred to Below

### 1.1.1.a Day 1—Preliminary Screening

An in vitro screening was performed with 70 Lactobacillus strains. The strains were selected on the basis of good survival properties under simulated intestinal-tract conditions, isolation from gastro-intestinal tract, or were strains from species that form the predominant Lactobacillus population in the gut.

Human buffy coat (containing a concentrated fraction of white blood cells) was obtained from the blood bank. Peripheral blood mononuclear cells were isolated by centrifugation over Ficoll-Paque. After washing, the cells were resuspended in RPMI 1640 medium containing 10% heat-inactivated fecal calf serum, 2 mM glutamine, and the antibiotics penicillin and streptomycin. Purified leukocytes were incubated in a volume of 2 ml at a concentration of 10⁶/ml. The final concentration of bacteria used in the assay was 10⁶ cfu/ml. Cells with bacteria were incubated in 24-well plates at 5% CO₂ and 37° C. for 24 h. Cytokines were determined in the culture supernatants using commercially available ELISA kits.

### 1.1.1.b Day 4—In Vitro Characterisation

The in vitro screening was carried out after Akdis C. A. et al (2003). Eur. J. Immunol. 33, pg 2717-2726. Blood cells were either not stimulated, or stimulated by LPS or by addition of anti-CD3 and anti-CD28 antibodies. Ratios of bacteria and blood cells were similar as in the one-day screening. Cytokines were determined by flow cytometric analysis, using labelled antibodies against the cytokines

### 1.1.2 Measurement of Cytokines

Cytokines measured on day 1 are intended to predict induction of Th1, Th2 or Treg. On the day 1 and day 4 characterisation, the following cytokines were measured:
IL-1β, as a marker for the pro-inflammatory response.
IFN-γ, as a marker for the Th1 response (not on day 1)
TNF-α, as a second marker for a pro-inflammatory response.
IL-12 (preferably, IL12p70), as a marker for a Th1 response.
IL-6, as a marker for a Th2 response (not on day 4).
IL-10, as a marker for T-regulatory cells (in combination with low IL-6)

### 1.2. Results

### 1.2.1 One-Day In Vitro Characterisation

The strains induced the stimulation of PBMC cytokine production, but as expected, there was a large amount of variation in cytokine levels. TNF-α was found in the highest amounts, followed by IL-6. These cytokines were all present in a range of approximately 10-40 ng/ml. IL-1β could be found in a concentration of around 4 ng/ml. IL-10 was present in a range of 0-1600 pg/ml and IL-12p70 was by far the lowest with a range of 0-200 pg/ml.

An interesting observation was that the immunomodulatory effect was to a large extent species dependent. For example, most L. plantarum strains were very effective in stimulation of IL-12 and TNF-α, whereas these cytokines were hardly stimulated by L. acidophilus strains.

Correlations between cytokines were studied in order to detect general characteristics of immune stimulation by lactobacilli. In this way, it was found that stimulation of IL-6 and IL-10 are generally correlated. IL-10 is increasingly produced after a threshold level of IL-6 (data not shown). Similarly, IL-12 production rapidly increases after a threshold value of TNF-α is exceeded (data not shown). At higher concentrations of TNF-α, the level of IL-1β has an unexpected maximum value of 4 to 4.5 ng/ml (data not shown).

Strains that are far from average are likely interesting for their immunomodulating potential. Three classes of strains can be distinguished:
1) strains that highly induce IL-12, but are low in IL-6 and IL-10 stimulation. These cells may specifically promote the Th1 response (data not shown).
2) strains that are high stimulators of IL-6 and IL-10 production and are suitable strains to promote the Th2 response (data not shown), and,
3) strains that either strongly induce IL-10 and/or that induce little of IL-12, IL-6 and/or the pro-inflammatory cytokines IL-1β and/or TNF-α are suitable strains for tolerance induction, i.e. the Th3 or T regulator response (data not shown).
   This is however a very preliminary subdivision that requires further confirmation in the 4-day cocultivation experiment below.

### 1.2.2 Four-Day In Vitro Characterisation

This screening was performed with 12 strains selected as described above. The aim was to identify the strains that were most powerful in stimulating both Th1 pathway and Treg (Th3) pathway. A PBMC cultivation experiment that lasted 4 days (compared to the 1 day experiment above) was used since the PBMCs get a better change to differentiate as a result of the stimulation. During a 4-day cultivation, cytokines are found that are produced by T-cells that have maturated during the cultivation period (Table 1). Therefore, the predictive value of this assay is more reliable than the 1-day assay performed above.

Another addition of this assay is that PBMCs were stimulated with either LPS (Table 2) or a mixture of antibodies recognizing the CD3 and CD28 leukocyte surface markers (Table 3). In this way, the influence of probiotic lactobacilli on the LPS or CD3/CD28 stimulation is studied.

Four strains (nrs 17, 26, 31 and 34) were identified as having the highest combination of IL-12, IFN-γ (both Th1 markers) and IL-10 (T-reg marker) inducers in. More specific, strains 17 and 26 are the highest Th1 cytokine inducers and also induce a reasonably high IL-10 whereas strains 31 and 34 are the highest IL-10 inducers that also result in reasonably high Th1 cytokine levels. The outcome is backed up by another Th1 marker (CD8) and T-reg marker (CD25). The other markers measured in this study relate to the level of stimulation, and proliferation of cells, and are less interesting for the selection of the strains.

These four strains correspond to the strains L. plantarum BI-1 (strain 17), L. plantarum BI-2 (strain 26), L. fermentum BI-6 (strain 31) and L. plantarum BI-3 (strain 34), respectively, and were deposited by NIZO Food Research, Ede, the Netherlands under the Treaty of Budapest on 18 Dec. 2006 at the Centraal Bureau voor Schimmelcultures, Baarn, The Netherlands. Deposited strains were assigned the following deposit no.'s: BI-1 Lactobacillus plantarum: CBS120663; BI-2 Lactobacillus plantarum: CBS120664; BI-6 Lactobacillus fermentum: CBS120661; and BI-3 Lactobacillus plantarum: CBS120662. BI-3 appeared to be initially incorrectly typed as a L. fermentum, but it was later re-typed.

TABLE 1
no stimulation day 4

| | IL-1B | IL-10 | IFN-γ | IL-12 |
|---|---|---|---|---|
| | (pg/ml) | (pg/ml) | (pg/ml) | (pg/ml) |
| Control | 337 | 368 | 156 | 0 |
| 5 | 503 | 794 | 243 | 7 |
| 6 | 333 | 685 | 2365 | 28 |
| 8 | 411 | 653 | 305 | 0 |
| 12 | 437 | 904 | 293 | 8 |
| 17 | 268 | 860 | 2225 | 63 |
| 26 | 289 | 787 | 1717 | 47 |
| 31 | 253 | 1233 | 942 | 16 |
| 34 | 333 | 1104 | 1103 | 27 |
| 44 | 160 | 787 | 903 | 29 |
| 64 | 213 | 609 | 2485 | 73 |
| 70 | 283 | 580 | 903 | 15 |
| 75 | 351 | 451 | 547 | 17 |

TABLE 2
stimulated by LPS day 4

| | IL-1B | IL-10 | IFN-γ | IL-12 |
|---|---|---|---|---|
| | (pg/ml) | (pg/ml) | (pg/ml) | (pg/ml) |
| Control | 201 | 375 | 132 | 0 |
| 5 | 238 | 895 | 195 | 0 |
| 6 | 180 | 734 | 1416 | 22 |
| 8 | 229 | 794 | 285 | 0 |
| 12 | 279 | 1092 | 257 | 0 |
| 17 | 160 | 802 | 2022 | 50 |
| 26 | 180 | 988 | 1465 | 41 |
| 31 | 165 | 1393 | 922 | 19 |
| 34 | 182 | 1393 | 858 | 24 |
| 44 | 121 | 1008 | 656 | 14 |
| 64 | 135 | 621 | 1340 | 33 |
| 70 | 166 | 615 | 459 | 10 |
| 75 | 201 | 487 | 421 | 8 |

TABLE 3
stimulated by anti-CD3/CD28 day 4

| | IL-1B | IL-10 | IFN-γ | IL-12 |
|---|---|---|---|---|
| | (pg/ml) | (pg/ml) | (pg/ml) | (pg/ml) |
| Control | 201 | 315 | 174 | 0 |
| 5 | 310 | 603 | 283 | 0 |
| 6 | 224 | 580 | 2485 | 30 |
| 8 | 280 | 487 | 376 | 0 |
| 12 | 289 | 692 | 293 | 0 |
| 17 | 193 | 580 | 3760 | 41 |
| 26 | 210 | 634 | 3247 | 40 |
| 31 | 176 | 877 | 1432 | 32 |
| 34 | 220 | 794 | 1840 | 37 |
| 44 | 160 | 692 | 1127 | 18 |
| 64 | 220 | 516 | 3811 | 48 |
| 70 | 236 | 451 | 942 | 9 |
| 75 | 298 | 421 | 729 | 15 |

### Example 2

### Effects of Lactobacilli on Cytokine Production by PBMC from Humans with Birch-Pollen Allergy

### 2.1 Experimental Design

Peripheral blood mononuclear cells (PBMC's) were harvested from blood donated by a healthy volunteer (P1), volunteers with birch-pollen allergy (P4, P6, P3, P5, P7 and P9) and volunteers with a non-related allergy (P2 and P8).

Cells were stimulated in different ways:
No stimulation by addition of plain growth medium
Non-specific stimulation of all T-cells with αCD3/αCD28. Primary analysis at day 4
Birch-pollen-specific stimulation with the main antigen Betyl, followed by stimulation with αCD3/αCD28 at day 7 (which only will affect cells that were already stimulated). Primary analysis at day 8. A smaller fraction of the cells will get activated and grow out. Therefore, cells are given more time to respond. Cytokines were determined by flow cytometric analysis, using labelled antibodies against the cytokines

The lactic acid bacterial strains included are listed in Table 4.

TABLE 4
Bacterial strains used in experiments of tables 5, 6 and 7

| Strain nr | Species |
|---|---|
| 8 | Lactobacillus acidophilus |
| 17 | Lactobacillus plantarum |
| 26 | Lactobacillus plantarum |
| 31 | Lactobacillus fermentum |
| 34 | Lactobacillus plantarum |
| 75 | Lactobacillus acidophilus |

### 2.2 Results

IL-13 is the major cytokine for inducing an allergic response. The data in Table 5 below illustrate that IL-13 is found when cells are specifically stimulated with Betyl (Table 5: row medium (Med)). The highest levels are found, as expected, in the volunteers that are allergic to birch pollen. The addition of lactic acid bacteria leads to an almost complete inhibition of IL-13 in most volunteers (except volunteer P7). The effect is limited or even absent when strains 8 or 34 are used, indicating that the effects are strain specific. The probiotic effect is also maintained when a mixture of strain 26 and 31 is included. Further data are acquired to elucidate the working mechanism for the inhibition of IL-13 formation.

IL-13 is a key cytokine produced by cells belonging to the T-helper 2 pathway of building up an immune response. A strategy to reduce this pathway is the stimulation of T-helper 1 pathway of which IL-12 is a major player. Indeed, the addition of probiotics 17, 26 and 31 compared to the medium control resulted in a stimulation of IL-12 production in CD3/CD28 stimulated PBMC's in 5 out of 6 birch pollen-allergic patients (see Table 6).

Another strategy to reduce IL-13 producing Th2 cells is believed to be the production IL-10 which is a cytokine produced by regulatory T-cells. As presented in Table 7 we find that in CD3/CD28 activated cells, IL-10 is stimulated by several of the bacterial strains (17, 26, 31 and 34) compared to the medium control.

In conclusion, we observed a birch-pollen specific reduction of IL-13 by specific lactic acid bacteria. This reduction is likely caused by a non-specific effect of probiotics on IL-10 and IL-12 production by PBMC's.

TABLE 5
Formation of I1-13 (pg/ml) in PBMC assays from three groups of volunteers: healthy
(P1) allergic (but not for birch pollen) (P2, P8) and allergic for birch pollen
(P4, P6, P3, P5, P7, P9): effect of different bacterial strains.

| | | No birch | | | | | | | |
|---|---|---|---|---|---|---|---|---|---|
| | Healthy | pollen allergy | | Birch pollen allergy | | | | | |
| Strain | P1 | P2 | P8 | P4 | P6 | P3 | P5 | P7 | P9 |
| No | 798 | 397 | 232 | 1586 | 1932 | 925 | 1460 | 1347 | 955 |
| (medium) | | | | | | | | | |
| 17 | 38 | 156 | 17 | 49 | 54 | 188 | 66 | 1180 | 75 |
| 26 | 34 | 137 | 20 | 29 | 87 | 106 | 55 | 724 | 83 |
| 31 | 46 | 149 | 10 | 38 | 52 | 212 | 31 | 529 | 30 |
| 26 + 31 | 43 | 120 | 8 | 24 | 50 | 76 | 22 | 529 | 45 |
| 8 | 141 | 547 | 95 | 267 | 111 | 690 | 335 | 1441 | 865 |
| 75 | 54 | 120 | 12 | 45 | 66 | 241 | 45 | 751 | 25 |
| 34 | | 287 | | 478 | 185 | | 376 | 769 | 483 |

TABLE 6
Formation of I1-12 (pg/ml) in PBMC assays from two groups
of volunteers: allergic (but not for birch pollen) (P2)
and allergic for birch pollen (P4, P6, P3, P5, P7, P9):
effect of different bacterial strains.

| | No birch | | | | | | |
|---|---|---|---|---|---|---|---|
| | pollen allergy | Birch pollen allergy | | | | | |
| Strain | P2 | P4 | P6 | P3 | P5 | P7 | P9 |
| No | 0 | 0 | 1 | 6 | 2 | 1 | 4 |
| (medium) | | | | | | | |
| 17 | 111 | 21 | 4 | 125 | 17 | 0 | 43 |
| 26 | 85 | 14 | 3 | 86 | 15 | 0 | 36 |
| 31 | 6 | 24 | 5 | 99 | 32 | 0 | 46 |
| 26 + 31 | 79 | 21 | 5 | 84 | 26 | 0 | 36 |
| 8 | 5 | 0 | 0 | 12 | 5 | 0 | 5 |
| 75 | 40 | 6 | 2 | 35 | 21 | 0 | 10 |
| 34 | 7 | 2 | 2 | | 10 | 0 | 7 |

TABLE 7
Formation of I1-10 (pg/ml) in PBMC assays from two groups
of volunteers: allergic (but not for birch pollen) (P2)
and allergic for birch pollen (P4, P6, P3, P5, P7, P9):
effect of different bacterial strains.

| | No birch | | | | | | |
|---|---|---|---|---|---|---|---|
| Probiotic | pollen allergy | Birch pollen allergy | | | | | |
| treatment | P2 | P4 | P6 | P3 | P5 | P7 | P9 |
| No | 96 | 161 | 58 | 97 | 72 | 140 | 32 |
| (medium) | | | | | | | |
| 17 | 102 | 198 | 84 | 124 | 109 | 347 | 38 |
| 26 | 127 | 214 | 85 | 137 | 128 | 500 | 49 |
| 31 | 106 | 216 | 87 | 117 | 120 | 490 | 40 |
| 26 + 31 | 114 | 190 | 96 | 120 | 101 | 516 | 44 |
| 8 | 87 | 354 | 52 | 122 | 56 | 319 | 32 |
| 75 | 63 | 98 | 42 | 68 | 53 | 188 | 9 |
| 34 | 149 | 484 | 136 | | 192 | 690 | 98 |

### Example 3

### Growth and Stability of the Selected Strains in Dairy Products

### 3.1 Growth and Stability in MRS and Milk

1 ml of 10% stock culture of cells in Mann Rogosa Sharp (MRS, Merck Company) medium (−40° C.) was mixed with 9 ml MRS. This was grown for 20 h at 37° C. The following media were inoculated with 1% of this culture: 1. MRS 2. milk. The milk was prepared as a 10% w/w solution of skim milk powder (Promex). The milk was sterilized for 10 min at 115°. After 24 h and 48 h the cultures were plated at MRS. Counting after 3 days growth at 37° C. Results are shown in Table 8.

After 24 h a high cell number is obtained in MRS, a lower amount in milk. However after 48 h cell numbers in MRS have decreased, in milk these are stable.

### 3.2 Stability in Yoghurt Like Product

Survival of the selected Lactobacillus strains in a yoghurt-like product environment was assessed under refrigeration conditions. Lactobacillus precultures were grown overnight in MRS. Protease positive Streptococcus thermophilus cultures used to prepare yoghurt like products were precultured overnight in milk treated for 10 minutes at 115° C. S. thermophilus precultures were inoculated at a 0.1% density together with a 1% inoculum of the Lactobacillus precultures in pasteurized (5 minutes at 85° C.) skim milk (prepared from milk powder) that had been cooled to 37° C. Inoculated cultures were aliquoted in portions of 100 ml. Skim milk cultures were grown at 37° C. for 15 hours and after growth were stirred with a sterile pipet, cooled to 4° C. and stored in a refrigerator (4-6° C.). Lactobacillus culture densities were determined by colony forming unit per ml enumeration on MRS-agar plates, directly after growth and cooling and after 28 days of storage in the refrigerator. The results as presented in Table 9 reveal clear differences between the different Lactobacillus strains. Although the densities of Lactobacillus cultures reached for each of the strains was within one log-unit difference after the 15 hours of growth the subsequent survival during storage of the yoghurt-like products differed markedly between the different strains.

It is interesting to compare the two L. acidophilis strains 5 and 70. In the one-day PBMC assay of Example 1 the immunomodulating patterns (low IL-12, high IL-10, comparatively low IL-6, indicating a high Treg capacity) of the 5 and 70 strains are quite similar, however Table 9 shows that their stability in yoghurt, and therefore the applicability in a fermented product differ greatly: strain 70 is much more stable than strain 5. This emphasises that one cannot just select on immunomodulating capacity but also the technological applicability (stability in a fermented product) has to be taken into account.

TABLE 8
Growth of cells in MRS and milk

| | | Cell numbers (cfu/ml) after growth in | | | |
|---|---|---|---|---|---|
| | | MRS | | Milk | |
| strain | Species | 24 h | 48 h | 24 h | 48 h |
| 5 | L. acidophilus | 4.4 × 10⁸ | 1.3 × 10⁸ | 2.9 × 10⁸ | 3.6 × 10⁸ |
| 8 | L. acidophilus | 8.1 × 10⁸ | 4.0 × 10⁸ | 9.6 × 10⁷ | 1.3 × 10⁸ |
| 17 | L. plantarum | 3.7 × 10⁹ | 4.9 × 10⁸ | 7.2 × 10⁸ | 1.7 × 10⁹ |
| 26 | L. plantarum | 3.6 × 10⁹ | 8.0 × 10⁷ | 1.3 × 10⁹ | 1.2 × 10⁹ |
| 31 | L. fermentum | 1.4 × 10⁹ | 1.5 × 10⁹ | 5.0 × 10⁸ | 1.0 × 10⁹ |
| 32 | L. fermentum | 7.2 × 10⁸ | 3.0 × 10⁶ | 4.2 × 10⁷ | 4.5 × 10⁷ |
| 34 | L. plantarum | 1.7 × 10⁹ | 1.4 × 10⁹ | 4.8 × 10⁸ | 6.0 × 10⁸ |
| 44 | L. acidophilus | 6.2 × 10⁸ | 2.4 × 10⁸ | 7.0 × 10⁷ | 8.4 × 10⁷ |
| 46 | L. reuteri | 1.4 × 10⁹ | 1.1 × 10⁸ | 4.0 × 10⁷ | 8.5 × 10⁷ |
| 62 | L. salivarius | 5.2 × 10⁸ | 4.5 × 10⁷ | 3.6 × 10⁸ | 6.1 × 10⁸ |
| 63 | L. salivarius | 7.8 × 10⁸ | 2.7 × 10⁸ | 7.5 × 10⁷ | 4.1 × 10⁷ |
| 64 | L. plantarum | 4.6 × 10⁹ | 7.0 × 10⁷ | 1.2 × 10⁹ | 1.6 × 10⁹ |
| 70 | L. acidophilus | 1.1 × 10⁹ | 1.2 × 10⁹ | 1.5 × 10⁸ | 2.3 × 10⁸ |

TABLE 9
Survival of strains in yoghurt-like product

| | | | Survival Log |
|---|---|---|---|
| | | | (cfu 28 days/ |
| Strains | Genus | Species | cfu 0 days) |
| 5 | Lactobacillus | acidophilus | −4.77 |
| 6 | Lactobacillus | rhamnosus | −0.08 |
| 8 | Lactobacillus | acidophilus | −1.94 |
| 12 | Lactobacillus | acidophilus | nd |
| 17 | Lactobacillus | plantarum | −1.27 |
| 26 | Lactobacillus | plantarum | −1.16 |
| 31 | Lactobacillus | fermentum | −0.08 |
| 34 | Lactobacillus | plantarum | −0.52 |
| 44 | Lactobacillus | acidophilus | −3.41 |
| 64 | Lactobacillus | plantarum | −0.78 |
| 70 | Lactobacillus | acidophilus | −1.47 |
| 75 | Lactobacillus | acidophilus | nd |

1-14. (canceled)

15. A lactic acid bacterium that upon in vitro co-incubation with human PBMCs induces one or more of:
a) at least 50% of the amount of IL-12; and,
b) at least 40% of the amount of IFN-γ;
as is induced under the same conditions in human PBMCs in vitro by at least one of four reference strains selected from L. plantarum BI-1, L. plantarum BI-2, L. fermentum BI-6 and L. plantarum BI-3.

16. A lactic acid bacterium according to claim 15, that further induces one or more of:
a) at least 50% of the amount of IL-10; and,
b) no more than 150% of the amount of IL-1β;
as is induced under the same conditions in human PBMCs in vitro by at least one of four reference strains selected from L. plantarum BI-1, L. plantarum BI-2, L. fermentum BI-6 and L. plantarum BI-3.

17. A lactic acid bacterium according to claim 15, wherein the bacterium is selected from strains L. plantarum BI-1, L. plantarum BI-2, L. fermentum BI-6 and L. plantarum BI-3.

18. A lactic acid bacterium according to claim 15, that further induces no more than 150% of the amount of IL-13 as is induced in vitro in human PBMCs from allergic individuals under the same conditions by at least one of the three reference strains selected from L. plantarum BI-1, L. plantarum BI-2 and L. fermentum BI-6.

19. A nutraceutical or pharmaceutical composition comprising a lactic acid bacterium according to claim 15.

20. A food product comprising a lactic acid bacterium according to claim 15.

21. The food product according to claim 9 wherein the food product is a dairy product selected from the group consisting of fermented dairy products, yoghurt, milk, milk-based drinks, buttermilk, yoghurt-based drinks, cheese, butter, ice-cream, a milk-derived dessert, soft curd cheese and fresh cheese.

22. The food product according to claim 20, wherein the product comprises:
a) a lactic acid bacterium that upon in vitro co-incubation with human PBMCs induces at least 50% of the amount of IL-12 and, at least 40% of the amount of IFN-γ as is induced under the same conditions by at least one of two reference strains selected from L. plantarum BI-1 and L. plantarum BI-2; and,
b) a lactic acid bacterium that upon in vitro co-incubation with human PBMCs induces at least 50% of the amount of IL-10, and preferably no more than 150% of the amount of IL-1β, as is induced under the same conditions by at least one of two reference strains selected from L. fermentum BI-6 and L. plantarum BI-3.

23. The food product according to claim 22, wherein the strain in a) is selected from L. plantarum BI-1 and L. plantarum BI-2, and wherein the strain in b) is selected from L. fermentum BI-6 and L. plantarum BI-3.

24. A method of treating an allergy in a mammal comprising administering to the mammal a composition comprising an effective amount of lactic acid bacterium according to claim 15.

25. The method according to claim 24, wherein the allergy is an IgE-mediated allergy.

26. The method according to claim 25, wherein the effective amount is between about 1×10⁶ to about 1×10¹² colony forming units per day.
